(19) United States
(12) Patent Application Publication
(10) Pub. No.: US 2017/0153216 A1
(43) Pub. Date: Jun. 1, 2017

(54) FACILITY MANAGEMENT SYSTEM

(71) Applicant: KURITA WATER INDUSTRIES LTD., Tokyo (JP)

(72) Inventors: Shintarou MORI, Tokyo (JP); Yukimasa SHIMURA, Tokyo (JP); Susumu FUKUE, Tokyo (JP)

(21) Appl. No.: 15/424,403

(22) Filed: Feb. 3, 2017

Related U.S. Application Data
(63) Continuation of application No. 14/893,387, filed on Nov. 23, 2015.

(30) Foreign Application Priority Data
Jun. 19, 2013 (JP) 2013-128640

(51) Int. Cl. G01N 33/18; C02F 1/00

(52) U.S. Cl. CPC G01N 33/18; C02F 1/008; C02F 2103/023

(57) ABSTRACT

A facility management system quickly creates information required for maintenance or the like of a water treatment facility, after water is sampled at the water treatment facility. The facility management system includes a management center including an operation database in which information indicating an operating status of a water treatment facility is stored, a portable water quality analyzer configured to perform water quality analysis on water taken from the water treatment facility, and a communication apparatus configured to acquire an analysis result from the portable water quality analyzer and wirelessly transmit the analysis result. The management center receives the analysis result transmitted from the communication apparatus, creates information indicating the operating status of the water treatment facility, by using the analysis result, and registers the information in the operation database.

### CROSS-REFERENCE TO RELATED APPLICATIONS

The present application is a continuation application of U.S. patent application Ser. No. 14/893,387, filed on Nov. 23, 2015, which is a National Phase Entry of International Application No. PCT/JP2014/062575, filed on May 12, 2014, which claims priority from Japanese Application No. 2013-128640, filed on Jun. 19, 2013, the disclosure of which is hereby incorporated by reference herein in its entirety.

### TECHNICAL FIELD

The present invention relates to a facility management system in which centralized management of an operating status of a water treatment facility or the like is performed at a management center.

### BACKGROUND ART

In a water treatment facility for a cooling water system or boiler water system, slime or scale is generated within a pipe by various impurities contained in treatment water. Thus, a chemical solution such as a slime inhibitor or a scale inhibitor is injected into a water system to be treated, in accordance with an operating status of the water treatment facility. Also, injection of the chemical solution is automatically controlled on the basis of information acquired by various sensors installed in the water treatment facility.

A management center of a facility management system periodically collects facility operation information of a water treatment facility to manage an operating status of the water treatment facility, analyzes the operating status of the water treatment facility in detail on the basis of the facility operation information or an analysis result of water to be treated, while referring to reference information stored in a database, creates advice information, maintenance information, or the like regarding a useful operation condition for operating the water treatment facility, and presents the advice information, the maintenance information, or the like to the water treatment facility side (see Patent Literature 1).

### CITATION LIST

### Patent Literature

PTL 1: Japanese Patent No. 3624941

### SUMMARY OF INVENTION

### Technical Problem

However, the existing facility management system described above requires at least a step of sampling water to be treated, from the water treatment facility, a step of transferring the sampled water to be treated, to an analysis center, a step of performing water quality analysis at the analysis center, and a step of registering a water quality analysis result together with a sampling date and time in a database, and takes a time period of about 1 week to 1 month from the time when the water is sampled to the time when the advice information, the maintenance information, or the like is presented to the water treatment facility side. Thus, even when maintenance of the water treatment facility or adjustment of the operation condition is performed on the basis of the presented information, it is difficult to efficiently operate the water treatment facility.

The present invention has been made in view of such a point, and an object of the present invention is to provide a facility management system and a program which are able to quickly create information required for maintenance or the like of a water treatment facility, after water is sampled at the water treatment facility.

### Solution to Problem

According to the present invention, a facility management system includes a management center including a first database in which information indicating an operating status of a water treatment facility is stored, a portable water quality analyzer configured to perform water quality analysis on water taken from the water treatment facility, and a communication apparatus configured to acquire an analysis result from the portable water quality analyzer and transmit the analysis result to the management center, wherein the management center receives the analysis result transmitted from the communication apparatus, creates information indicating the operating status, by using the analysis result, and registers the information in the first database.

According to one aspect of the present invention, the management center includes a second database in which facility information of each of a plurality of the water treatment facilities is stored, the facility information including at least one of a location, a name, a type, and specifications, and a transmission unit configured to transmit the facility information to the communication apparatus.

According to one aspect of the present invention, the facility information includes specifications of transmission of the analysis result, and the communication apparatus transmits the analysis result to the management center on the basis of the specifications of transmission included in the facility information received from the management center.

According to one aspect of the present invention, the communication apparatus includes a display unit, acquires the information indicating the operating status from the management center, and displays the acquired information on the display unit.

According to one aspect of the present invention, the management center includes a third database in which a correspondence relationship between each of a plurality of users who use the portable water quality analyzer and the water treatment facility for which each user performs water quality analysis is specified, the transmission unit transmits the correspondence relationship to the communication apparatus, and the communication apparatus accepts identification information of the user who uses the portable water quality analyzer, and displays the facility information of the water treatment facility for which the user corresponding to the identification information performs water quality analysis, on the display unit on the basis of the correspondence relationship.

According to one aspect of the present invention, the communication apparatus notifies the management center of the identification information, and the transmission unit transmits the information indicating the operating status of the water treatment facility for which the user corresponding to the identification information performs water quality analysis, to the communication apparatus on the basis of the correspondence relationship.

According to one aspect of the present invention, the portable water quality analyzer includes a storage unit configured to store the analysis result, acquires the facility information from the communication apparatus, and writes the facility information into the storage unit such that the facility information is linked to the analysis result, and the communication apparatus acquires the analysis result linked to the facility information, from the water quality analyzer.

According to one aspect of the present invention, the management center includes a data analysis unit configured to detect a problem in the water treatment facility or make the analysis result into a trend graph, by using the analysis result received from the communication apparatus, and the problem detected by the data analysis unit or the trend graph created by the data analysis unit is registered in the first database as the information indicating the operating status.

### Advantageous Effects of Invention

According to the present invention, it is possible to quickly create information required for maintenance or the like of the water treatment facility, after water is sampled by the water treatment facility, so that it is possible to efficiently operate the water treatment facility.

### BRIEF DESCRIPTION OF DRAWINGS

FIG. 1 is a schematic configuration diagram of a facility management system according to an embodiment.

FIG. 2 is a hardware configuration diagram of a communication apparatus according to the embodiment.

FIG. 3 is a diagram of functional blocks realized by execution of a facility management program according to the embodiment.

FIG. 4 is a flowchart for explaining a facility management method according to the embodiment.

### DESCRIPTION OF EMBODIMENTS

Hereinafter, an embodiment of the present invention will be described based on the drawings.

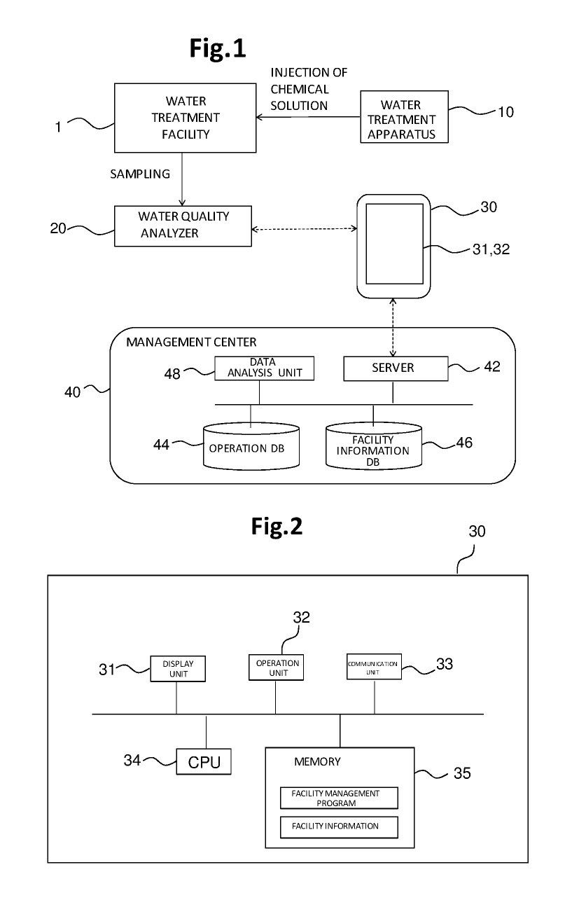

FIG. 1 is a schematic configuration diagram of a facility management system according to the present embodiment. As shown in FIG. 1, the facility management system includes: a water treatment apparatus 10 which performs a predetermined treatment on a water treatment facility 1; a water quality analyzer 20 which analyzes the water quality of water taken (sampled) from the water treatment facility 1; a communication apparatus 30 which acquires an analysis result from the water quality analyzer 20 and wirelessly transmits the analysis result; and a management center 40 provided with an operation database 44 in which the analysis result transmitted from the communication apparatus 30 is stored. The facility management system manages an operating status of the water treatment facility 1.

The water treatment facility 1 is a cooling tower in this embodiment. Cooling water discharged from the cooling tower, water circulating in the cooling tower, or water to be supplied to the cooling tower is sampled and the water quality thereof is analyzed by the water quality analyzer 20.

The water treatment apparatus 10 injects a chemical solution such as a slime inhibitor or a scale inhibitor into water passing through the cooling tower as the water treatment facility 1, to prevent a cooling water system from being dirty or to prevent a pipe from being corroded.

The water quality analyzer 20 analyzes at least one item of water quality items such as electric conductivity, pH, temperature, and chemical concentration of the water sampled from the water treatment facility 1. The water quality analyzer 20 may analyze two or more of the items. The water quality analyzer 20 is a portable small-sized water quality analyzer (portable water quality analyzer), and is able to analyze the water quality of sampled water near the water treatment facility 1. The water quality analyzer 20 may have a waterproof structure so as to allow the water quality analyzer 20 to perform measurement in water.

The communication apparatus 30 acquires a water quality analysis result from the water quality analyzer 20 and transmits the water quality analysis result to the management center 40. The communication apparatus 30 is a wireless communication apparatus which includes a display unit 31 and an operation unit 32. For example, a smartphone or a tablet type personal computer which includes a touch panel forming the display unit 31 and the operation unit 32 may be used as the communication apparatus 30. In acquiring an analysis result from the water quality analyzer 20, the communication apparatus 30 may performs wired communication via a serial communication cable such as RS-232C or may perform wireless communication via a wireless LAN.

The management center 40 includes: a server 42 which transmits and receives data to and from the communication apparatus 30; and the operation database 44 in which the analysis result transmitted from the communication apparatus 30 is stored. In the case where there are a plurality of water treatment facilities 1 to be managed, the management center 40 is provided with a facility information database 46 in which facility information which specifies the installation location, name, type, specifications, and the like of each water treatment facility is stored. An example of the facility information is shown in Table 1 below.

TABLE 1

| Customer | | Facility | | | Transmission |
|---|---|---|---|---|---|
| code | Customer name | code | Facility name | Water type | CSV file name |
| ABCDEFG001 | ◯◯Company□▪ factory | ABCD001 | Cooling tower | Make-up water | UploadFile001.csv |
| | | | No. 1 | | |
| ABCDEFG001 | ◯◯Company□▪ factory | ABCD001 | Cooling tower | Cooling water | UploadFile002.csv |
| | | | No. 1 | | |
| ABCDEFG001 | ◯◯Company□▪ factory | ABCD002 | Cooling tower | Cooling water | UploadFile003.csv |
| | | | No. 2 | | |
| ABCDEFG001 | ◯◯Company□▪ factory | ABCD003 | Cooling tower | Cooling water | UploadFile004.csv |
| | | | No. 3 | | |
| ABCDEFG001 | ◯◯Company□▪ factory | ABCD004 | Cooling tower | Cooling water | UploadFile005.csv |
| | | | No. 4 | | |
| HIJKLMN123 | ΔΔCompanyXX factory | K000000 | Cooling tower A | Make-up water | UploadFile006.csv |
| HIJKLMN123 | ΔΔCompanyXX factory | K000000 | Cooling tower A | Cooling water | UploadFile007.csv |
| K888888888 | ⋄⋄Company▾▾factory | K000000 | Cooling tower | Make-up water | UploadFile008.csv |
| K888888888 | ⋄⋄Company▾▾factory | K000000 | Cooling tower | Cooling water | UploadFile009.csv |
| . . . | . . . | . . . | . . . | . . . | . . . |

In the above Table 1, “customer code” and “customer name” correspond to the installation location. “Facility code” and “facility name” correspond to the name of the water treatment facility. “Water type” corresponds to the type of water to be subjected to water quality analysis.

“Transmission CSV file name” specifies data transmission specifications for the communication apparatus 30 to transmit the analysis result to the management center 40.

The communication apparatus 30 acquires, from the water quality analyzer 20, an analysis result of “make-up water” for “cooling tower No. 1” installed at the customer code “ABCDEFG001” creates a CSV file which has a name of “UploadFile001” and includes this analysis result, and transmits the CSV file to the server 42 of the management center 40.

The server 42 determines a data registration destination within the operation database 44 on the basis of the file name of data received from the communication apparatus 30, the customer code or the facility code recorded in the file, and registers the analysis result in the operation database 44, as information indicating the operating status of the water treatment facility 1. The information is registered in the operation database 44 in a form shown in Table 2 below.

TABLE 2

| | | | | | | | | Date of |
|---|---|---|---|---|---|---|---|---|
| Date of | Time of | Customer | Facility | | | Electric | | sensor |
| analysis | analysis | code | code | Water type | pH | conductivity | Temperature | calibration |
| 20120531 | 11:00 | ABCDEFG001 | ABCD004 | Cooling water | 7.0 | 10.0 | 22.0 | 20120517 |
| 20120429 | 10:00 | HIJKLMN1231 | K000000 | Cooling water | 6.0 | 20.0 | 25.0 | 20120428 |

The information indicating the operating status of the water treatment facility 1 may further include a start/stop signal, a water volume, a water pressure, and the like of the water treatment facility 1.

As shown in FIG. 1, the management center 40 is provided with a data analysis unit 48 which performs analysis, diagnosis, and processing for display on the data registered in the operation database 44. The data analysis unit 48 averages an analysis result, or calculates an index value of a trend diagnosis (rising/lowering speed calculation) or the like. The data analysis unit 48 compares the calculated index value with a threshold (a value for determining presence/absence of a problem) to detect a problem in the water treatment facility 1. The data analysis unit 48 performs data processing such as making time-series data into a trend graph so as to allow the data to be displayed in a display unit of another apparatus. An analytical result and a diagnosis result by the data analysis unit 48, the created trend graph, and the like are stored in the operation database 44, as information indicating the operating status of the water treatment facility 1.

The server 42 transmits the analytical result, the diagnosis result, the trend graph, and the like stored in the operation database 44, to the communication apparatus 30 in accordance with a request from the communication apparatus 30. The communication apparatus 30 displays the trend graph or the like received from the server 42, on the display unit 31.

Accordingly, for example, while referring to the trend graph displayed on the display unit 31, a person in charge of water treatment is allowed to adjust an amount of a chemical solution to be injected by the water treatment apparatus 10, or to maintain the water treatment facility 1 in a stable and optimum operation condition by requesting a person in charge of facility to adjust the facility load of the water treatment facility 1.

FIG. 2 shows an example of the hardware configuration of the communication apparatus 30. The communication apparatus 30 includes the display unit 31, the operation unit 32, a communication unit 33, a CPU (central processing unit) 34, and a memory 35.

The display unit 31 is composed of a liquid crystal display or the like, and displays the analysis result, the analytical result, the diagnosis result, the trend graph, and the like acquired from the server 42.

The operation unit 32 accepts various instructions from a user. In addition, the operation unit 32 accepts an ID (identification information) of the user who uses the communication apparatus 30, or a password for using the communication apparatus 30.

The communication unit 33 includes a wireless communication unit which wirelessly transmits and receives data to and from the server 42. The communication unit 33 includes a wired communication unit which performs wired communication with the water quality analyzer 20.

The memory 35 stores a facility management program to be executed by the CPU 34, and the facility information received from the server 42 of the management center 40. The memory 35 is composed of, for example, a NAND and/or NOR flash memory.

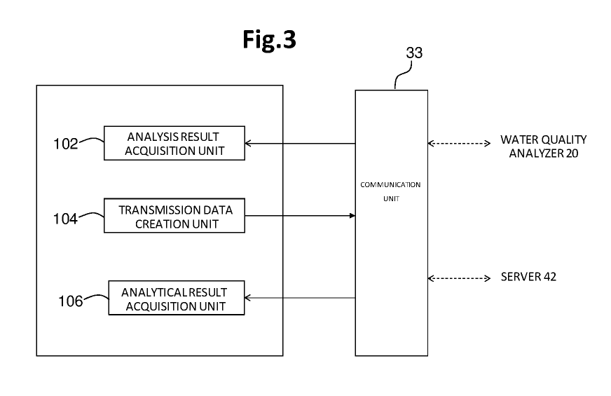

The CPU 34 executes the facility management program in the memory 35. FIG. 3 shows a diagram of functional blocks realized by executing the facility management program. By execution of the facility management program, an analysis result acquisition unit 102, a transmission data creation unit 104, and an analytical result acquisition unit 106 are realized.

The analysis result acquisition unit 102 acquires the analysis result from the water quality analyzer 20 via the communication unit 33.

The transmission data creation unit 104 creates transmission data including the analysis result acquired by the analysis result acquisition unit 102, such that the transmission data has a form instructed by the management center 40, on the basis of the data transmission specifications included in the facility information. The created transmission data is wirelessly transmitted via the communication unit 33 to the server 42 of the management center 40.

The analytical result acquisition unit 106 acquires the analytical result, the diagnosis result, the trend graph, and the like, which are created by the data analysis unit 48, via the communication unit 33 from the server 42. The analytical result acquisition unit 106 writes the acquired data into the memory 35.

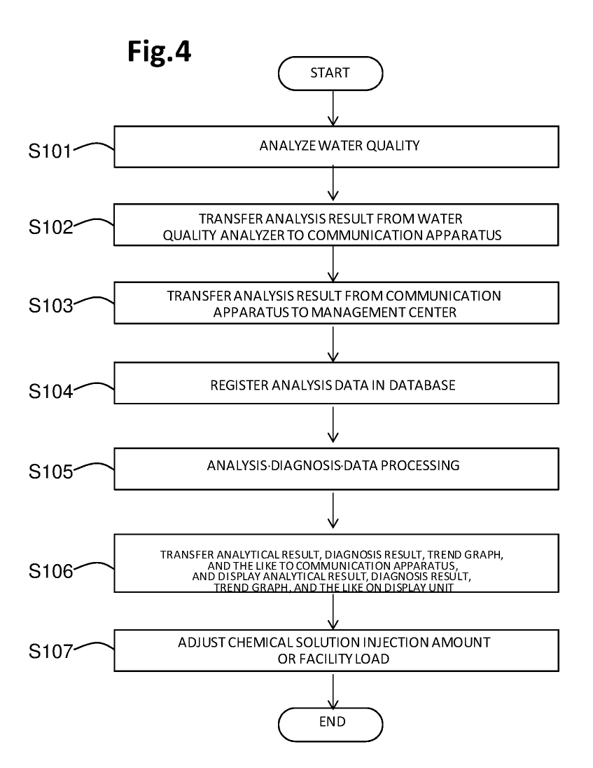

Next, a method for managing a water treatment facility by using the facility management system according to the present embodiment will be described by using a flowchart shown in FIG. 4. The communication apparatus 30 acquires the facility information from the management center 40 beforehand.

[Step S101] The user analyzes the water quality of water sampled from the water treatment facility 1, by using the water quality analyzer 20. The analysis result is stored into a memory of the water quality analyzer 20. Since the water quality analyzer 20 is a portable small-sized analyzer, the water quality analyzer 20 is able to perform water quality analysis near the water treatment facility 1.

[Step S102] The water quality analyzer 20 and the communication apparatus 30 are connected via a wire or wirelessly to each other, and the analysis result acquired in step S101 is transferred from the water quality analyzer 20 to the communication apparatus 30. The communication apparatus 30 stores the analysis result acquired from the water quality analyzer 20, into the memory 35.

Here, before the analysis result is transferred, the facility information is displayed on the display unit 31, and a water treatment facility and a water type subjected to the water quality analysis are designated by using the operation unit 32. The analysis result transferred from the water quality analyzer 20 is linked to the designated water treatment facility and water type and is stored into the memory 35.

[Step S103] The communication apparatus 30 transmits the analysis result acquired in step S102, to the server 42 of the management center 40. Specifically, the transmission data creation unit 104 creates transmission data including the analysis result, on the basis of data transmission specifications corresponding to the water treatment facility and the water type designated by means of the operation unit 32 in step S102. Then, the communication unit 33 transmits the transmission data.

[Step S104] The server 42 receives the data from the communication apparatus 30. The server 42 determines a data registration destination within the operation database 44 on the basis of the file name of the data received from the communication apparatus 30, the customer code or the facility code registered in the file, and registers the analysis result in the operation database 44, as information indicating the operating status of the water treatment facility 1. By periodically performing steps S101 to S104, a plurality of analysis results of waters of which sampling times are different are stored into the operation database 44.

[Step S105] The data analysis unit 48 of the management center 40 performs analysis, diagnosis, creation of a trend graph for display, and the like by using the analysis results registered in the operation database 44. An analytical result, a diagnosis result, the created trend graph, and the like are stored into the operation database 44.

[Step S106] The communication apparatus 30 acquires, from the management center 40, the analytical result, the diagnosis result, the trend graph, and the like which are created by the data analysis unit 48 in step S105.

For example, the operation unit 32 accepts, from the user, an instruction to acquire a trend graph of a water quality analysis result. On the basis of the instruction accepted by the operation unit 32, the analytical result acquisition unit 106 sends a request of transmitting the trend graph, to the server 42 via the communication unit 33. On the basis of the transmission request sent from the communication apparatus 30, a transmission unit of the server 42 takes out the trend graph from the operation database 44 and transmits the trend graph to the communication apparatus 30. The communication apparatus 33 displays the trend graph received from the server 42, on the display unit 31.

In this manner, it is possible to display the analytical result, the diagnosis result, the trend graph, and the like created by the data analysis unit 48, on the display unit 31.

[Step S107]

The person in charge of treatment adjusts an amount of the chemical solution to be injected by the water treatment apparatus 10, or requests the person in charge of facility to adjust the facility load of the water treatment facility 1, while referring to the trend graph displayed on the display unit 31.

According to the present embodiment, it is not necessary to send water sampled from the water treatment facility 1, to the analysis center, and it is possible to perform water quality analysis near the water treatment facility 1, so that it is possible to acquire an analysis result quickly after the water is sampled.

The analysis result acquired by the water quality analyzer 20 is transferred to the communication apparatus 30, is wirelessly transmitted from the communication apparatus 30 to the server 42 of the management center 40, and is registered in the operation database 44 by the server 42. Thus, it is possible to quickly register the analysis result in the operation database 44.

By the data analysis unit 48 performing analysis and diagnosis of data, creation of a trend graph, and the like upon registration of a new analysis result in the operation database 44, it is possible to quickly create an analytical result, a diagnosis result, a trend graph, and the like in which the latest water quality analysis result is reflected.

The communication apparatus 30 is able to acquire the analytical result, the diagnosis result, the trend graph, and the like created by the data analysis unit 48, via wireless communication, and display the analytical result, the diagnosis result, the trend graph, and the like. As described above, according to the present embodiment, it is possible to make the time period from the time when water is sampled by the water treatment facility 1 to the time when the trend graph or the like in which the latest water quality analysis result is reflected, to be very short. By adjusting an amount of the chemical solution to be injected by the water treatment apparatus 10 or adjusting the facility load of the water treatment facility 1 while referring to the trend graph or the like displayed on the display unit 31 as described, it is possible to maintain the water treatment facility 1 in a stable and optimum operation condition.

In the above embodiment, the communication apparatus 30 links the water quality analysis result to the water treatment facility and the water type, but the water quality analyzer 20 may link the water quality analysis result to the water treatment facility and the water type. For example, beforehand, the facility information is transmitted from the communication apparatus 30 to the water quality analyzer 20 and stored into the memory of the water quality analyzer 20. Then, a water treatment facility and a water type subjected to water quality analysis are designated on the water quality analyzer 20, and a water quality analysis result is linked to the water treatment facility and the water type. The communication apparatus 30 acquires, from the water quality analyzer 20, the water quality analysis result which is linked to the water treatment facility and the water type.

The communication apparatus 30 may acquire a water quality analysis result linked to a water treatment facility and a water type, every single water quality analysis, or may collectively acquire a plurality of pieces of data after water quality analysis is performed for a plurality of water treatment facilities or water types. Similarly, the communication apparatus 30 may create transmission data and transmit the transmission data to the server 42 each time acquiring a single analysis result, or may create a plurality of pieces of transmission data corresponding to a plurality of analysis results, and collectively transmit the transmission data to the server 42.

The management center 40 may be provided with a database in which a correspondence relationship between each of a plurality of users who use the water quality analyzer 20 and the water treatment facility 1 for which each user performs water quality analysis is specified. The transmission unit of the server 42 transmits the correspondence relationship to the communication apparatus 30. Upon acceptance of the ID of the user who uses the water quality analyzer 20 and the communication apparatus 30, the communication apparatus 30 displays the facility information of the water treatment facility for which the user corresponding to the accepted ID performs water quality analysis, on the display unit 31 on the basis of the correspondence relationship. Thus, the user is allowed to easily recognize the water treatment facility for which the user performs water quality analysis.

Upon acceptance of the ID of the user who uses the water quality analyzer 20, the communication apparatus 30 may prohibit display of the facility information of the water treatment facilities for which the users other than the user corresponding to the accepted ID perform water quality analysis, on the basis of the above-described correspondence relationship. Thus, each user is not able to access the facility information of the water treatment facilities handled by the other users, so that it is possible to improve information security.

Each user may be allowed to acquire an analytical result, a diagnosis result, a trend graph, and the like created by the data analysis unit 48 for only the water treatment facility handled by the user. For example, in sending a request of transmitting a trend graph or the like to the server 42, the communication apparatus 30 notifies the server 42 of the ID of the user who uses the communication apparatus 30. The server 42 transmits, to the communication apparatus 30, only the trend graph or the like which is viewable by the user corresponding to the notified ID, on the basis of the above-described correspondence relationship between each of the plurality of users who use the water quality analyzer 20 and the water treatment facility 1 for which each user performs water quality analysis. Thus, each user is not able to view the trend graphs or the like of the water treatment facilities handled by the other users, so that it is possible to further improve information security.

The facility management program described in the above embodiment may be distributed via a communication line (including wireless communication) such as the Internet. The program may be distributed in an encrypted, modulated, or compressed state via a wireless line or a wired line such as the Internet, or may be stored in a storage medium and distributed.

The above embodiment is an example of the present invention, and the present invention may be implemented in an embodiment other than the above.

The present invention has been described in detail by using the specific modes, but it is obvious to a person skilled in the art that various changes may be made without departing from the intention and scope of the present invention.

1. A facility management system comprising:
a management center including a first database in which information indicating an operating status of a water treatment facility is stored, a second database in which facility information of each of a plurality of the water treatment facilities is stored, the facility information including at least one of a location, a name, a type, and specifications, and a transmission unit;
a portable water quality analyzer configured to perform water quality analysis on water taken from the water treatment facility; and
a communication apparatus configured to acquire an analysis result from the portable water quality analyzer and transmit the analysis result to the management center,
wherein the management center receives the analysis result transmitted from the communication apparatus, creates information indicating the operating status, by using the analysis result, and registers the information in the first database,
the transmission unit of the management center transmits the facility information to the communication apparatus,
the facility information includes specifications of transmission, which contain a file name, of the analysis result, and
the communication apparatus transmits the analysis result to the management center on a basis of the specifications of transmission included in the facility information received from the management center.

2. The facility management system according to claim 1, wherein the communication apparatus includes a display unit, acquires the information indicating the operating status from the management center, and displays the acquired information on the display unit.

3. The facility management system according to claim 2, wherein the management center includes a third database in which a correspondence relationship between each of a plurality of users who use the portable water quality analyzer and the water treatment facility for which each user performs water quality analysis is specified,
the transmission unit transmits the correspondence relationship to the communication apparatus, and
the communication apparatus accepts identification information of the user who uses the portable water quality analyzer, and displays the facility information of the water treatment facility for which the user corresponding to the identification information performs water quality analysis, on the display unit on the basis of the correspondence relationship.

4. The facility management system according to claim 3, wherein the communication apparatus notifies the management center of the identification information, and
the transmission unit transmits the information indicating the operating status of the water treatment facility for which the user corresponding to the identification information performs water quality analysis, to the communication apparatus on the basis of the correspondence relationship.

5. The facility management system according to claim 1, wherein the portable water quality analyzer includes a storage unit configured to store the analysis result, acquires the facility information from the communication apparatus, and writes the facility information into the storage unit such that the facility information is linked to the analysis result, and
the communication apparatus acquires the analysis result linked to the facility information, from the water quality analyzer.

6. The facility management system according to claims 1, wherein the management center includes a data analysis unit configured to detect a problem in the water treatment facility or make the analysis result into a trend graph, by using the analysis result received from the communication apparatus, and
the problem detected by the data analysis unit or the trend graph created by the data analysis unit is registered in the first database as the information indicating the operating status.
